SEQUENCE LISTING (8 sequences)

SEQ ID NO: 1 - RNA, 21 nt, synthetic construct
  source 1..21: mol_type = other RNA
gcaaagggaataagaaatttt

SEQ ID NO: 2 - RNA, 21 nt, synthetic construct
  source 1..21: mol_type = other RNA
aatttcttattccctttgctt

SEQ ID NO: 3 - RNA, 21 nt, synthetic construct
  source 1..21: mol_type = other RNA
gcctcgactatcacgggattt

SEQ ID NO: 4 - RNA, 21 nt, synthetic construct
  source 1..21: mol_type = other RNA
atcccgtgatagtcgaggctt

SEQ ID NO: 5 - RNA, 21 nt, synthetic construct
  source 1..21: mol_type = other RNA
gcagccactgatttcaaattt

SEQ ID NO: 6 - RNA, 21 nt, synthetic construct
  source 1..21: mol_type = other RNA
atttgaaatcagtggctgctt

SEQ ID NO: 7 - RNA, 21 nt, synthetic construct
  source 1..21: mol_type = other RNA
ttctccgaacgtgtcacgttt

SEQ ID NO: 8 - RNA, 21 nt, synthetic construct
  source 1..21: mol_type = other RNA
acgtgacacgttcggagaatt

(19) United States
(12) Patent Application Publication
(10) Pub. No.: US 2026/0139252 A1
(43) Pub. Date: May 21, 2026

(54) NANOCOMPLEX TARGETING NERVOUS NECROSIS VIRUS AS WELL AS PREPARATION METHOD AND USE THEREOF

(71) Applicant: Yazhou Bay Innovation Institute, Hainan Tropical Ocean University, Sanya (CN)

(72) Inventors: Jielan JIANG, Sanya (CN); Mingguang MAO, Sanya (CN); Yunxiang MAO, Sanya (CN)

(73) Assignee: Yazhou Bay Innovation Institute, Hainan Tropical Ocean University, Sanya (CN)

(21) Appl. No.: 19/432,957

(22) Filed: Dec. 25, 2025

Related U.S. Application Data
(63) Continuation of application No. PCT/CN2025/103758, filed on Jun. 26, 2025.

(30) Foreign Application Priority Data
Nov. 15, 2024 (CN) 202411635937.6

(51) Int. Cl. C12N 15/113; A61P 31/14

(52) U.S. Cl. CPC C12N 15/1131; A61P 31/14; C12N 2310/14; C12N 2310/351

(57) ABSTRACT

The present disclosure relates to the technical field of prevention and control of diseases in aquatic animals, and particularly relates to a nanocomplex targeting a nervous necrosis virus as well as a preparation method and use thereof. The present disclosure provides a nanocomplex composed of poly(lactic-co-glycolic acid), polyethyleneimine and siRNA. The present disclosure further provides a preparation method of the nanocomplex and use of the nanocomplex in the preparation of a drug for improving the survival rate of fish eggs infected with NNV. In the present disclosure, a vector for an NNV capsid protein is constructed by simulating the NNV; the constructed NNV capsid protein is expressed in cells; the siRNA capable of effectively inhibiting the expression of the viral capsid protein is screened, and then the nanocomplex of the NNV is constructed; and the NNV is blocked in the embryonic stage (fertilized eggs).

### CROSS-REFERENCE TO RELATED APPLICATIONS

The application claims priority to Chinese patent application No. 202411635937.6, filed on Nov. 15, 2024, the entire contents of which are incorporated herein by reference.

### SEQUENCE LISTING

The sequence listing xml file submitted herewith, named “P-701US-2500096.xml”, created on Dec. 4, 2025, and having a file size of 8,192 bytes, is incorporated by reference herein.

### TECHNICAL FIELD

The present disclosure relates to the technical field of prevention and control of diseases in aquatic animals, and particularly relates to a nanocomplex targeting a nervous necrosis virus as well as a preparation method and use thereof.

### BACKGROUND

Although significant breakthroughs have been achieved in the artificial propagation of marine fish in China, relatively few species have realized large-scale artificial propagation. Moreover, mass mortality of fry often occurs in the early stages of artificial propagation, which has become one of the major bottlenecks restricting the scale of marine fish aquaculture and stock enhancement. One of the causes of mass early-stage mortality in marine fish is a nervous necrosis virus (NNV) which was discovered in 1989 and first reported in 1990. After that, it was successively reported worldwide that more than 100 species of marine fish carried NNV, over 40 species of them are susceptible to the virus, e.g., Dicentrarchus labrax, Lates calcarifer, Ephinephelus spp., Pseudocaranx dentex, Solea senegalensis, Hippoglossus hippoglossus, Gadus morhua, Gadus macrocephalus, etc., (Bandin I and Souto S., 2020). NNV primarily occurs in marine fish during the larval, juvenile and fry stages, with the mortality rate of up to 100%. Currently, there are no effective methods to block NNV at home and abroad, which has remained a major bottleneck plaguing marine fish seedling rearing.

Studies have shown that one of the main propagation routes of NNV is vertical propagation from parents to offspring. Both eggs and sperm may carry the virus. Before the hatching of fertilized eggs, the embryos are protected by the egg membranes and the operation is convenient, so this period is an ideal period to block NNV. Post-hatching fish larvae have weak stress resistance in water, and therefore are not suitable for drug application. Therefore, blocking NNV starting from the embryonic stage can effectively avoid the adverse effects of NNV on fish larvae. There are many drugs for blocking viruses, e.g., vaccines, small interfering RNA (siRNA), interferons, etc. The application of RNA interference technology (RNAi) in the treatment of virus-induced infectious diseases has attracted considerable attention (Zuoyan Zhu et al., 2006), however, there are no effective methods to deliver these drugs to fish embryos currently. Therefore, there is an urgent need to develop an effective and feasible drug delivery carrier.

With the continuous innovation of novel technical means in the field of medicines, delivery systems based on various materials such as proteins, liposomes, polymers and inorganic materials have been applied in research on efficient siRNA delivery, with significant progress in targeted tumor diagnosis and therapy. However, there is little research on siRNA nanodelivery technologies in the field of aquaculture.

### SUMMARY

In view of this, the technical problem to be solved by the present disclosure is to provide a nanocomplex targeting a nervous necrosis virus as well as a preparation method and use thereof. The nanocomplex provided in the present disclosure is capable of effectively increasing the survival rate of fish eggs, and can be effectively applied to the field of aquaculture.

The present disclosure provides a nanocomplex, wherein the nanocomplex is composed of the following raw materials: poly(lactic-co-glycolic acid), polyethyleneimine and siRNA, wherein
a mass-to-volume ratio of the poly(lactic-co-glycolic acid) to the polyethyleneimine is (5-50) mg:(0.05-1) mL, and each 100 mL of the polyethyleneimine contains 0.5-2.5 mol of siRNA;
the siRNA comprises at least one of a positive-sense strand of a nucleotide sequence as shown in SEQ ID NO: 1 and a negative-sense strand of a nucleotide sequence as shown in SEQ ID NO: 2, a positive-sense strand of a nucleotide sequence as shown in SEQ ID NO: 3 and a negative-sense strand of a nucleotide sequence as shown in SEQ ID NO: 4, and a positive-sense strand of a nucleotide sequence as shown in SEQ ID NO: 5 and a negative-sense strand of a nucleotide sequence as shown in SEQ ID NO: 6; and
the nanocomplex has a particle size of 100-200 nm.

Compared with the prior art, in the nanocomplex provided in the present disclosure, the poly(lactic-co-glycolic acid) has good biocompatibility, no toxicity and excellent encapsulation and film-forming properties, and its degradation products are lactic acid and hydroxyacetic acid which are also byproducts of a human metabolic pathway, and therefore it has no toxic and side effects when applied to pharmaceuticals and biological materials; the siRNA is capable of effectively inhibiting NNV, while the polyethyleneimine can better form the complex with the siRNA, thereby further providing the load of the poly(lactic-co-glycolic acid); and the components cooperate closely to successfully construct the NNV-targeting nanocomplex which can effectively enter the interior of fish eggs and block NNV in the embryonic stage, thereby improving the survival rate of fish fry and achieving more accurate technical effects.

In some embodiments, the mass-to-volume ratio of the poly(lactic-co-glycolic acid) to the polyethyleneimine is 25 mg: 0.1 mL, and each 100 mL of the polyethyleneimine contains 1.25 mol of siRNA, wherein
the molecular weight of the poly(lactic-co-glycolic acid) is 10000 Da, and the molecular weight of the polyethyleneimine is 25000 Da.

In some embodiments, the siRNA comprises the positive-sense strand of the nucleotide sequence as shown in SEQ ID NO: 1 and the negative-sense strand of the nucleotide sequence as shown in SEQ ID NO: 2.

In some embodiments, the nanocomplex has the particle size of 160 nm.

Experiments have demonstrated that the nanocomplex prepared using respective components described in the aforementioned embodiments exhibit the most intimate synergistic interaction among all components, along with optimal loading capacity and particle size. This enables superior delivery efficiency into fish eggs, thereby achieving the most accurate technical effects.

The present invention further provides a preparation method of the nanocomplex, comprising the following steps:
Step 1, mixing the siRNA with the polyethyleneimine to obtain a solution A, and mixing poly(lactic-co-glycolic acid) with an organic phase solution to obtain a solution B;
Step 2, performing ultrasonic mixing on the solution A and the solution B in Step 1;
Step 3, performing ultrasonic mixing on the solution subjected to ultrasonic mixing in Step 2 and an external aqueous phase solution again; and
Step 4, performing filtration and centrifugation on the solution subjected to ultrasonic mixing again in Step 3 so as to obtain the nanocomplex.

In some embodiments, in the Step 1, a solvent used for mixing the solution A comprises water.

Preferably, the concentration of the polyethyleneimine in the solution A is 5 vol %-50 vol %.

More preferably, the concentration of the polyethyleneimine in the solution A is 10 vol %.

In some embodiments, in the Step 1, the organic phase solution comprises chloroform and/or dimethylformamide.

Preferably, the organic phase solution is chloroform.

Preferably, the concentration of the poly(lactic-co-glycolic acid) in the solution B is 1-10 mg/mL.

In some embodiments, the concentration of the poly(lactic-co-glycolic acid) can be 1 mg/mL, 2 mg/mL, 3 mg/mL, 4 mg/mL, 5 mg/mL, 6 mg/mL, 7 mg/mL, 8 mg/mL, 9 mg/mL or 10 mg/mL.

More preferably, the concentration of the poly(lactic-co-glycolic acid) in the solution B is 5 mg/mL.

In some embodiments, in the Step 3, the solute of the external aqueous phase solution comprises polyethylene glycol and/or Tween 80.

Preferably, the solute of the external aqueous phase solution is polyethylene glycol.

Preferably, the concentration of polyethylene glycol in the external aqueous phase solution is 1-20 mg/mL.

More preferably, the concentration of polyethylene glycol in the external aqueous phase solution is 10 mg/mL.

In some embodiments, in the Step 2 and Step 3, a volume ratio of the solution A to the solution B to the external aqueous phase solution is (0.5-2):(1-10):(1-30).

More preferably, the volume ratio of the solution A to the solution B to the external aqueous phase solution is 1:5:15.

In some embodiments, in the Step 4, the filtration adopts ultrafiltration, and after the centrifugation, the Step 4 further comprises washing with PBS to obtain the nanocomplex.

The present disclosure provides use of the nanocomplex or the nanocomplex obtained by using the preparation method in the preparation of a drug for resisting NNV and/or improving the survival rate of fish eggs infected with NNV.

The present disclosure provides a drug for resisting a nervous necrosis virus (NNV) and/or improving the survival rate of fish eggs infected with NNV, comprising the nanocomplex or the nanocomplex obtained by using the preparation method.

The present disclosure provides a method for improving the survival rate of fish eggs infected with NNV, comprising performing medicated bath using the drug.

The present disclosure provides the nanocomplex composed of the poly(lactic-co-glycolic acid), the polyethyleneimine and the siRNA. The present disclosure further provides the preparation method of the nanocomplex and use of the nanocomplex in the preparation of the drug for resisting NNV and/or improving the survival rate of fish eggs infected with NNV. In the present disclosure, a vector for an NNV capsid protein is constructed by simulating the NNV; the constructed NNV capsid protein is expressed in cells; the siRNA capable of effectively inhibiting the expression of the viral capsid protein is screened, and then the nanocomplex of the NNV is constructed; and the NNV is blocked in the embryonic stage, thereby improving the survival rate of fish fry and further opening up new possibilities for the treatment of early-stage diseases in fish fry. The method has innovation in the field of artificial propagation of aquatic animals and provides a novel idea for viral prevention and control in the aquaculture industry.

### BRIEF DESCRIPTION OF THE DRAWINGS

FIG. 1-FIG. 16 illustrate co-transfection of Epithelioma papulosum cyprini (EPC) cells with siRNA and pEGFP-CP at different transfection time points (×100), wherein FIG. 1 shows pEGFP-CP at 24 h; FIG. 2 shows pEGFP-CP+NNV-18 at 24 h; FIG. 3 shows pEGFP-CP+NNV-144 at 24 h; FIG. 4 shows pEGFP-CP+NNV-720 at 24 h; FIG. 5 shows pEGFP-CP at 48 h; FIG. 6 shows pEGFP-CP+NNV-18 at 48 h; FIG. 7 shows pEGFP-CP+NNV-144 at 48 h; FIG. 8 shows pEGFP-CP+NNV-720 at 48 h; FIG. 9 shows pEGFP-CP at 72 h; FIG. 10 shows pEGFP-CP+NNV-18 at 72 h; FIG. 11 shows pEGFP-CP+NNV-144 at 72 h; FIG. 12 shows pEGFP-CP+NNV-720 at 72 h; FIG. 13 shows pEGFP-empty vector at 24 h; FIG. 14 shows pEGFP-empty vector at 48 h; FIG. 15 shows pEGFP-empty vector at 72 h; and FIG. 16 shows siRNA-Cy3 at 48 h.

FIG. 17 illustrates the cell viabilities of EPC in different control groups. In the figure, * represents that this group exhibits significant difference compared with a control group (P<0.05), ** represents extremely significant difference (P<0.01), and ns represents no significant difference.

FIG. 18 illustrates the cell viability of EPC after co-transfection with siRNA and pEGFP-CP. In the figure, * represents that this group shows significant difference compared with a control group (P<0.05), and ** represents extremely significant difference (P<0.01).

FIG. 19 illustrates electron microscopic observation results of a nanocomplex in a filter membrane after secondary ultrasonic treatment of an optimal ratio group followed by ultrafiltration in Example 2.

FIG. 20 illustrates fluorescence detection results of a nanocomplex in a filter membrane after secondary ultrasonic treatment of an optimal ratio group followed by ultrafiltration in Example 2.

FIG. 21 illustrates the effect of a nanocomplex in a filter membrane after secondary ultrasonic treatment of an optimal ratio group followed by ultrafiltration in Example 2 on grouper eggs. In the figure, Ctr is a control group; C1 is a 10-fold dilution concentration; and C2 is a 1000-fold dilution concentration.

### DETAILED DESCRIPTION OF THE EMBODIMENTS

The present disclosure provides a nanocomplex targeting a nervous necrosis virus as well as a preparation method and use thereof. Those skilled in the art can appropriately adjust the process parameters to implement the present disclosure based on the contents herein. It is especially noted that all similar replacements and variations are obvious for those skilled in the art, and shall be deemed to be included within the present disclosure. The method and use of the present disclosure have been described through preferred embodiments, the method and use herein can be altered, or appropriately changed or combined without departing from the contents, spirits and ranges of the present disclosure to implement and apply the present disclosure.

It should be noted that relational terms such as first and second are used herein merely to distinguish one entity or operation from another entity or operation and do not necessarily require or imply the existence of any such actual relationship or sequence between these entities or operations. Moreover, the terms “include”, “comprise” or any other variants thereof are intended to cover non-exclusive inclusion, so that a process, method, items or apparatus comprising a series of elements includes not only those elements but also other elements not explicitly listed, or further includes inherent elements of such process, method, item or apparatus.

Verified by preliminary experiments, compared with other nanomaterials, the nanocomplex selected in the present disclosure exhibits the highest compatibility with fish fertilized eggs. It has an appropriate particle size and does not block the pores and channels of fish eggs, ensuring normal material exchange; it possesses stable chemical properties, does not release toxic substances, and reduces interference with the internal biochemical processes of fish eggs; it is not prone to inducing oxidative stress, decreases the production of reactive oxygen species, and can protect intracellular biological macromolecules; it has no endocrine-disrupting effects, maintaining the normal hormonal signal transduction and developmental process of fish eggs; it has minimal impact on the gene expression of fish eggs, ensuring the normal expression of development-related genes and promoting normal development and differentiation. Therefore, the present invention adopts the poly(lactic-co-glycolic acid) and polyethyleneimine-loaded siRNA to construct the nanocomplex. The preliminary experiment has poor effect, and therefore cannot be temporarily provided in the present application.

When the nanocomplex formed by siRNA and nanomaterials poly(lactic-co-glycolic acid) and polyethyleneimine in a ratio selected in the present disclosure, various components are closely coordinated and have optimal load capacity. Furthermore, the successfully constructed nanocomplex has the best particle size, which is more conducive to entry into the fish eggs, and then improving the survival rate of the fish fry by blocking the NNV in the embryo stage.

Test materials used in the present disclosure are all common commercially available products, and can be purchased from the market. Next, the present invention will be further illustrated in detail in conjunction with embodiments.

### Example 1 Screening of siRNA Interference Sequence

### 1. Materials and Methods

### (1) Synthesis of Simulated Virus

RGNNV is simple in structure, and only has a structural protein, i.e., capsid (CP). A gene encoding CP was inserted into a cell transfection vector pEGFP so as to lay a solid foundation for subsequent stimulation of this virus in cells.

(2) Design of siRNA Interference Sequence

To realize the effective silencing of the NNV CP gene, the siRNA sequence was designed utilizing an online tool provided by Ambion company (http://www.ambion.com) based on the sequence of the NNV CP gene that had existed in a laboratory. During the design, the selected siRNA sequence was ensured to be completely complementary to mRNA of the target CP gene to realize specific gene silencing. Through online basic local alignment search tool (BLAST), the selected sequence was ensured to have no homology to other genes except this virus. Meanwhile, to verify the specificity of siRNA, a negative control (siRNA-Cy3, siRNA labeled with red fluorescence dye Cy3) was designed in this experiment. All the sequences were entrusted to Shanghai GenePharma Co., Ltd. for synthesis.

TABLE 1
siRNA sequence

| | | Target | SEQ | |
|---|---|---|---|---|
| | | fragment | ID | |
| Name | Sequence 5′-3′ | length (bp) | NO | Target |
| NNV- | GCAAAGGGAAUAAGAAAUUTT | 21 | 1 | siRNA in |
| 18-F | | | | experimental |
| | | | | group |
| NNV- | AAUUUCUUAUUCCCUUUGCTT | 21 | 2 | siRNA in |
| 18-R | | | | experimental |
| | | | | group |
| NNV- | GCCUCGACUAUCACGGGAUTT | 21 | 3 | siRNA in |
| 144-F | | | | experimental |
| | | | | group |
| NNV- | AUCCCGUGAUAGUCGAGGCTT | 21 | 4 | siRNA in |
| 144-R | | | | experimental |
| | | | | group |
| NNV- | GCAGCCACUGAUUUCAAAUTT | 21 | 5 | siRNA in |
| 720-F | | | | experimental |
| | | | | group |
| NNV- | AUUUGAAAUCAGUGGCUGCTT | 21 | 6 | siRNA in |
| 720-R | | | | experimental |
| | | | | group |
| siRNA- | UUCUCCGAACGUGUCACGUTT | 21 | 7 | siRNA in |
| Cy3-F | | | | control group |
| siRNA- | ACGUGACACGUUCGGAGAATT | 21 | 8 | siRNA in |
| Cy3-R | | | | control group |

Note:
there was no base U in characters used in a nucleotide in the sequence listing, and therefore when the sequence listing was made, the base U in the RNA sequence in Table 1 described above was changed into base T.

(3) Inhibitory Effect of siRNA on Intracellular CP
Bacterial Culture with Shaking and Plasmid Extraction (Endotoxin-Free Plasmid Mini-Prep Kit)

(1) an LB liquid culture medium was prepared in a ratio, an Escherichia coli bacterial solution cryopreserved at −20° C. was taken and then 200 μL of ice-thawed Escherichia coli bacterial solution was aspirated into the culture medium, and subsequently the culture medium was put in a shaker after sealing and labeling, where the temperature was set as 37° C., and the rotation speed was set as 200 rpm.

(2) The cultured bacteria were centrifuged (generally centrifuged for 5-10 min at 4° C. at about 4000-6000 g). Supernatant was discarded, and bacterial pellets were retained.

(3) A lysis buffer provided by a kit was added to the bacterial pellets to be sufficiently and evenly mixed, so that the bacteria were lysed (standing for a period of time at room temperature to promote the lysis).

(4) An equal volume of neutralization buffer was added to the aforementioned lysate to be evenly mixed, and then subjected to standing at room temperature to neutralize the active ingredients in the lysate.

(5) The mixture in a centrifugal tube was generally centrifuged for 10-15 min at 4° C. at about 12000-14000 g. The supernatant was discarded, and pellets containing plasmid DNA were retained.

(6) A wash buffer was added to the pellets to be evenly mixed, and then the obtained mixture was centrifuged to remove the wash buffer. The wash step was repeated at least once to ensure the removal of impurities such as all proteins and RNA.

(7) A certain volume of elution buffer (generally sterile water or TE buffer) was added to the final pellets. The above materials were evenly mixed, and then the tube was placed at room temperature or at 37° C. for incubation to sufficiently elute the plasmid DNA.

(8) The eluted plasmid DNA was transferred to a new sterile tube and stored at −20° C. or −80° C. for later use in subsequent experiments.

### Earlier Culture of EPC Cells:

(1) The activated EPC cells were cultured in an M199 culture medium containing 20% fetal bovine serum, and the growth condition of cells was determined according to the adhesion status of the cells.

(2) When the cells have confluent growth on the bottom of a culture flask, a waste liquid in a biosafety cabinet was removed, trypsin was added for digestion, the flask was gently tapped to disperse the cells, then the flask wall was pipetted using an M199 medium containing 5% fetal bovine serum, and whether passage was feasible was determined according to the cell density.

(3) When in passage, a half of the liquid was aspirated after digestion and transferred to a new culture flask. The growth status of the cells was observed every 2-3 days, and the culture medium was exchanged to ensure sufficient nutrients and promote the growth of the cells.

### Design of Experimental Group:

(1) (Blank) control group 1: cells without any treatment.

(Negative) control group 2: only pEGFP-empty vector plasmids were transfected. The plasmids were empty vector plasmids with green fluorescence.

(Positive) control group 3: only pEGFP-CP was transfected. The plasmid was a plasmid with a CP pathogenic gene and green fluorescence.

(Negative) control group 4: only siRNA-Cy3 was transfected. siRNA-Cy3 was siRNA labeled with red fluorescence dye Cy3, and had no homology to the CP sequence.

Experimental group 1: co-transfection of pEGFP-CP and NNV-18.

Experimental group 2: co-transfection of pEGFP-CP and NNV-144.

Experimental group 3: co-transfection of pEGFP-CP and NNV-720.

### Cell Transfection Experiment:

By taking a 24-well plate as an example, at 18-24 h prior to the transfection experiment, 0.5-2×10⁵ cells were inoculated into each well, and 0.5 mL of cell culture medium was added for culture.

The quantity of cells for earlier inoculation was selected, which enabled the cell density to reach 70%-90% when in transfection, and it was noted that during the transfection, the cells were ensured not to be overgrown or in a quiescent phase.

25 μL of Opti-MEM culture medium was taken and added to two 1.5 mL sterile EP tubes respectively, and the two tubes were labeled as tube 1 and tube 2 respectively.

Lipofectamin 3000 transfection reagent was placed at room temperature and gently and evenly mixed before use; 0.75 μL of Lipofectamin 3000 transfection reagent was added to tube 1 containing a serum-free culture medium, the above materials were evenly mixed by pipetting up and down, and then the obtained mixture was placed for 5 min at room temperature.

1 μg (the required volume was calculated based on the desired amount) of NNV plasmid/empty vector was added to tube 2, the above materials were evenly mixed by gently pipetting up and down. Then, 1 μL of P3000™ Reagent was added for mixing, and the materials were evenly mixed by gently pipetting up and down. 0.625 μL of transfection siRNA was added for transfection of siRNA, without the addition of the P3000™ Reagent.

A transfection complex was added to the Lipofectamin 3000 mixture, i.e., 25 μL of mixture in tube 2 was added to tube 1, the above materials were evenly mixed by pipetting up and down and then subjected to standing for 10-15 min at room temperature, to facilitate DNA/siRNA and the Lipofectamin 3000 reagent to form the transfection complex.

50 μL of complex was added to a culture plate containing cells (a plasmid complex and a siRNA complex were respectively added to co-transfection, with 100 μL in total), and then the culture plate was gently tapped and shaken to evenly disperse the cells. 3 wells were set up in each group.

The culture plate was placed in a cell incubator at 25° C. for culture of 24 h, 48 h and 72 h, respectively.

### Cell Observation and Counting:

At different transfection time points, the growth status of the cells in the culture plate was observed under an inverted microscope, and photographing was taken under a fluorescence microscope and the fluorescence signals were recorded. The microscope was set to 100× magnification (10× objective lens×10× eyepiece), and 5-8 fields of view were observed in each experimental well to ensure a comprehensive evaluation of the transfection efficiency.

The supernatant in the culture plate was collected and transferred to a new sterile EP tube, and each well in the culture plate was washed with PBS.

200 μL of trypsin was added in each well to digest the cells for 2 min by gently tapping, 300 μL of culture solution was added, the above materials were then evenly mixed by gently tapping, the obtained mixture was added in an EP tube containing the supernatant, 500 μL of trypan blue solution was then added, and then the materials were evenly mixed by pipetting up and down.

25 μL of cell suspension was aspirated from the tube, and then placed in a counting chamber for counting using a cell counter.

### Data Analysis:

The statistical significance of differences among groups was analyzed using GraphPad Prism v.8.0.2 and graphs are plotted. All trials were performed in triplicate, and data comparison was conducted using two-way analysis of variance (ANOVA). *:P<0.05; **: P<0.01; ns: no significant difference.

### 2. Results and Analysis

### 2.1. Fluorescence Observation in Each Group

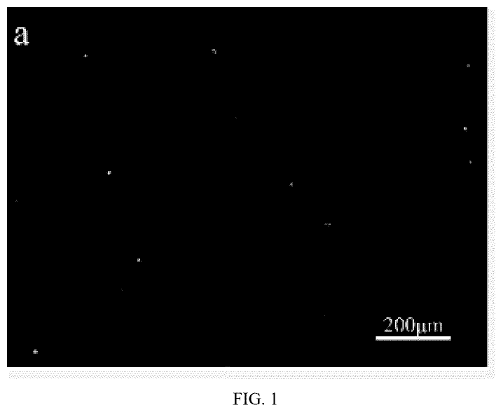

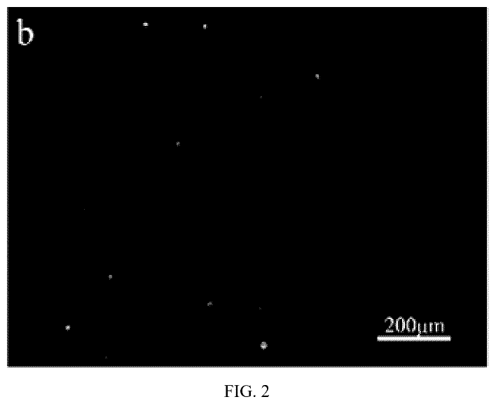

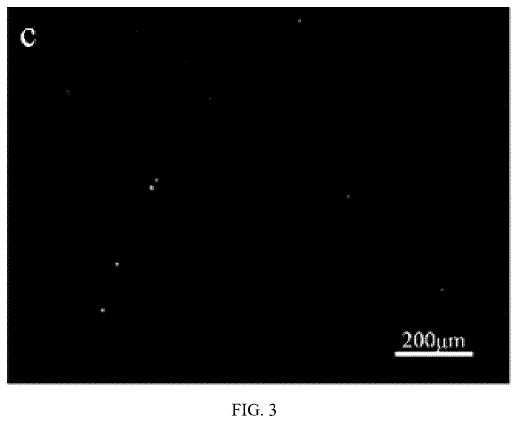

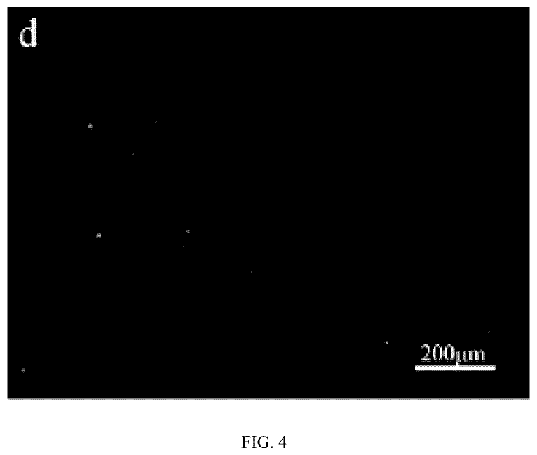

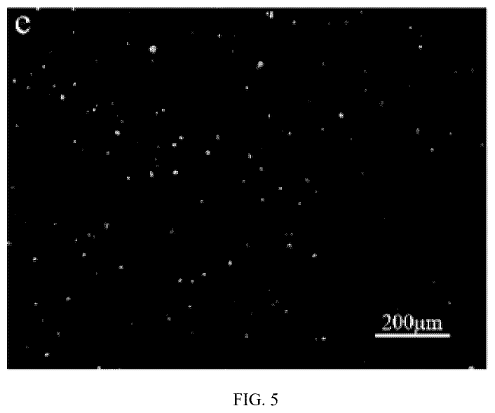

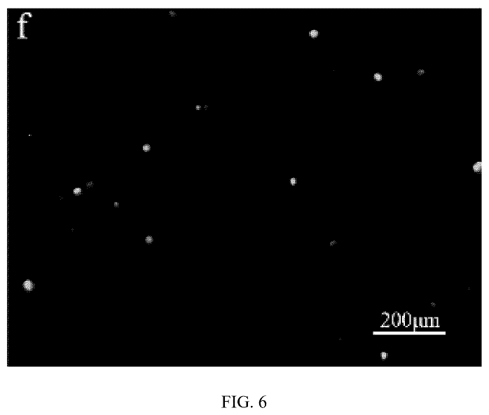

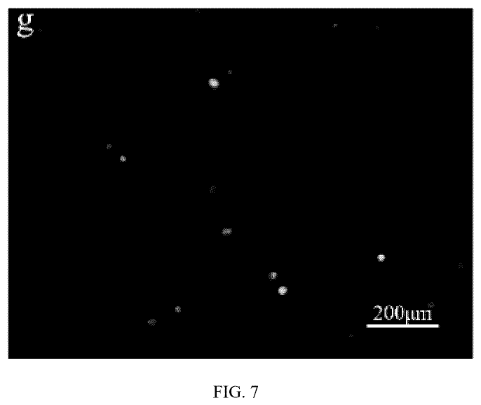

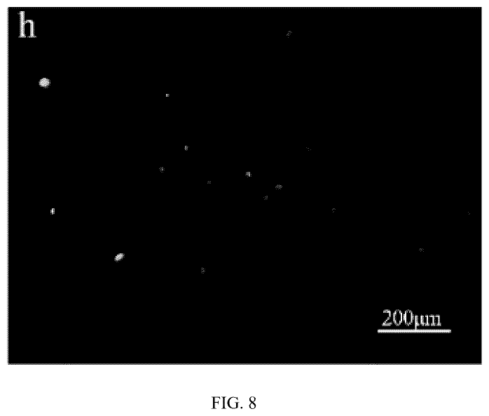

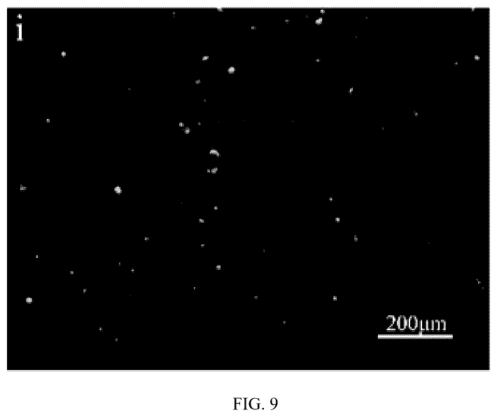

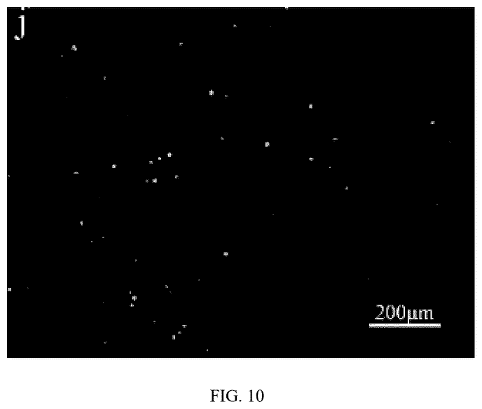

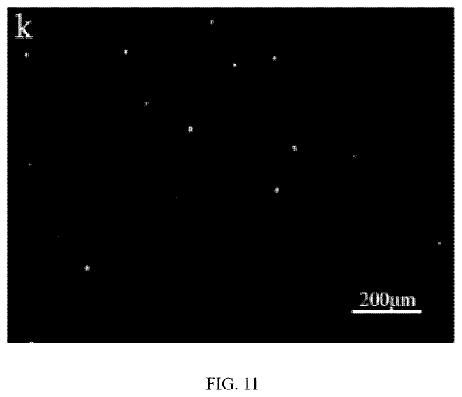

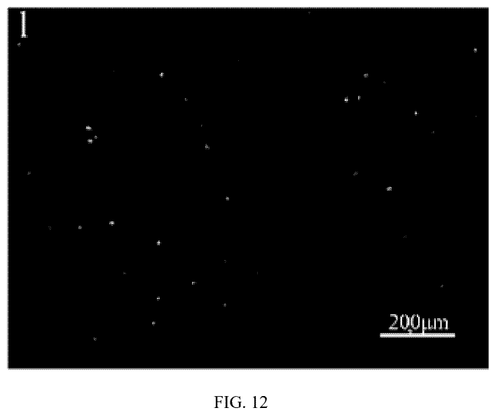

At different transfection time points (24 h, 48 h and 72 h), negative control groups solely transfected with pEGFP-CP (as shown in FIG. 1, FIG. 5 and FIG. 9) exhibited a large number of green fluorescent signals, with the highest intensity at 48 h. Furthermore, the successfully transfected cells displayed a round morphology. The experimental groups co-transfected with pEGFP-CP and siRNA exhibited fewer green fluorescent signals that were all fewer than those in positive control groups at corresponding time points. Especially at the transfection time point of 48 h, the green fluorescent signals in pEGFP-CP and siRNA co-transfected groups were obviously reduced (as shown in FIG. 2, FIG. 3, FIG. 4, FIG. 6, FIG. 7, FIG. 8, FIG. 10, FIG. 11 and FIG. 12).

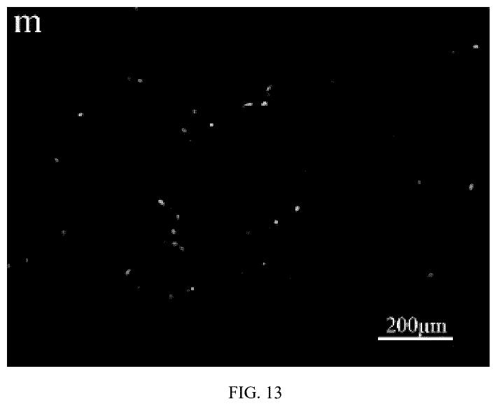

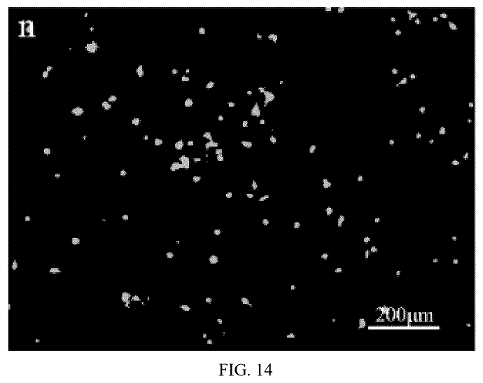

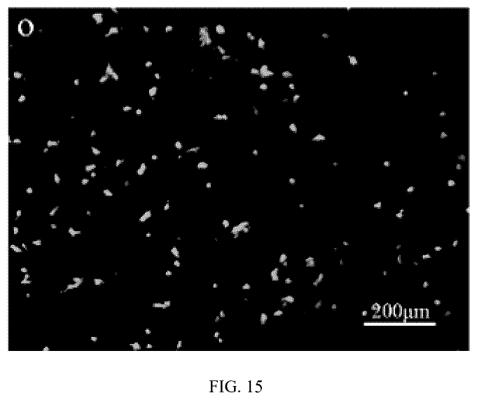

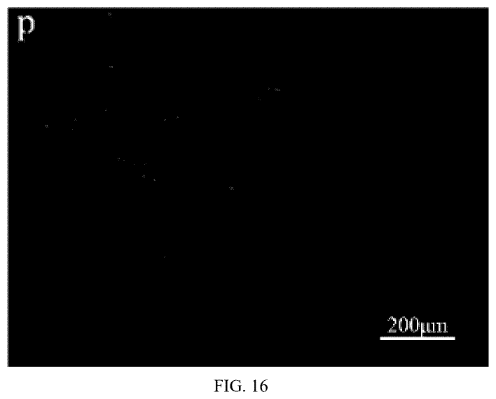

In addition, the fluorescent signals in the pEGFP-empty vector group gradually increased over time, with irregularly shaped green cells observed under the microscope (as shown in FIG. 13, FIG. 14 and FIG. 15). Red fluorescent signals were detectable in the siRNA-Cy3-transfected experimental groups (as shown in FIG. 16).

### 3.2. EPC Counting

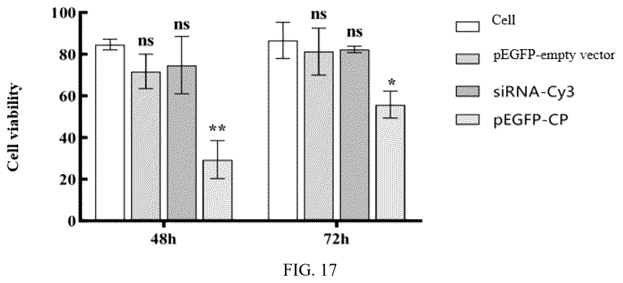

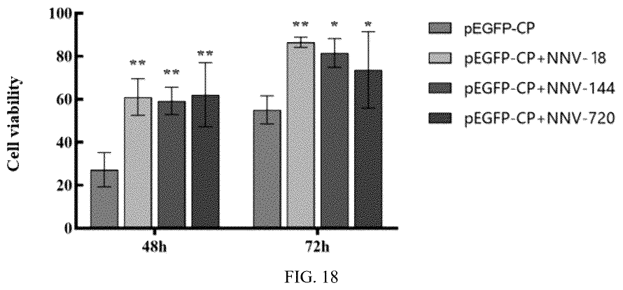

The cell viabilities of EPC in different control groups were as shown in FIG. 17. The cell viabilities of the control group, pEGFP-empty vector group and the siRNA-Cy3 group at 48 h were 84.55%, 71.69% and 74.69%, respectively, and the cell viabilities of the pEGFP-empty vector group and the siRNA-Cy3 group at 72 h were 86.60%, 81.24% and 82.23%, respectively. Moreover, compared with the cell control group, both the pEGFP-empty vector group and the siRNA-Cy3 group exhibited no significant difference compared with the cell control group. Compared with the cell control group, the pEGFP-CP positive control group exhibited extremely significant difference at 48 h, and significant difference at 72 h. The cell viability of EPC after co-transfection of siRNA and pEGFP-CP is as shown in FIG. 18. In the figure, the cell viabilities of EPC after co-transfection of NNV-18, NNV-144, NNV-720 and pEGFP-CP for 48 h and 72 h obviously increased, indicating that these 3 pairs of siRNA all exhibit inhibitory effects on the expression of CP, with relatively small cell toxic effects. At 48 h post-transfection, the cell viability of the pEGFP-CP group was about 27%, the cell viabilities of pEGFP-CP+NNV-18, pEGFP-CP+NNV-144 and pEGFP-CP+NNV-720 groups were 61%, 59% and 62%, respectively. The pEGFP-CP control group exhibited extremely significant difference (P<0.01), and there is no significant difference among the three groups. At 72 h post-transfection, the cell viabilities of the pEGFP-CP control group, the pEGFP-CP+NNV-18 group, the pEGFP-CP+NNV-144 group and the pEGFP-CP+NNV-720 group were 55%, 86%, 81% and 73%, respectively, all of which increased compared with those at 48 h. Among these groups, the pEGFP-CP+NNV-18 group exhibited extremely significant difference compared with the pEGFP-CP control group (P<0.01), the pEGFP-CP+NNV-144 group, the pEGFP-CP+NNV-720 group exhibited significant difference compared with the pEGFP-CP control group (P<0.05), while there was no significant difference among the pEGFP-CP+NNV-18 group, the pEGFP-CP+NNV-144 group and the pEGFP-CP+NNV-720 group.

The above-mentioned results indicated that the simulated virus can cause relatively high cell lethality, while the cell viability after addition of siRNA can obviously increase, indicating that these siRNA can effectively inhibit the viruses. The NNV-18 group had the best siRNA effect, and was used for subsequent preparation of the nanocomplex.

### Example 2 Preparation of PLGA-siRNA Nanocomplex

### 1. Synthesis of PLGA-siRNA Nanocomplex

(1) Organic phase: 5 mg of PLGA with a molecular weight of 10000 was dissolved into different volumes of organic solvents (chloroform or dimethyl formamide (DMF)), with an optimal ratio screened, to form the organic phase.

(2) Internal aqueous phase: PEI (polyethyleneimine) with a molecular weight of 25000 was dissolved into water, with an optimal ratio screened; and siRNA was dissolved into a PEI aqueous solution at 4° C. to form the internal aqueous phase.

(3) External aqueous phase: PEG or Tween80 was dissolved into water, with an optimal ratio selected, so as to form the external aqueous phase.

(4) The organic phase and the internal aqueous phase were mixed in a ratio, followed by first ultrasonic mixing using an ultrasonic apparatus.

(5) The solution subjected to ultrasonic treatment was added to the prepared external aqueous phase in a ratio, and then the above mixed solution was subjected to secondary ultrasonic treatment.

(6) After the ultrasonic treatment, a certain amount of solution was centrifuged, and then supernatant was taken. The supernatant was added in an ultrafiltration tube for centrifugation, a spherical complex coated with siRNA was filtered to a filter membrane, then the nanocomplex on the filter membrane was suspended into PBS or an aqueous solution using PBS or the aqueous solution, and then the obtained suspension was stored at a low temperature in the dark.

(7) The particle size and zeta potential of the product subjected to secondary ultrasonic treatment were measured using a nanoparticle laser particle size analyzer, and the final product was observed via an electron microscope.

In the steps as described above, the concentration of PEI, the concentration of PLGA, the volume-to-molar mass ratio of polyethyleneimine to siRNA, the main solutes and their respective concentrations in the organic phase solution and the external aqueous phase solution, as well as the volume ratio of the internal aqueous phase to the organic phase solution to the external aqueous phase solution were prepared in groups according to Table 2. Following the operational modification based on the above-described synthesis, the particle size of the product subjected to secondary ultrasonic treatment was measured. The optimal reagents were compared based on particle size distribution and dilutable ratios. In a dilutable ratio test, 2 mL of PLGA-siRNA nanocomplexes prepared in Groups A1-A4 of Table 2 were subjected into logarithmic serial dilution with deionized water, and the optimal preparation product was obtained based on the maximum ratio at which the final product remained stable.

TABLE 2
Different grouping conditions

| | | | | | | A volume ratio of |
|---|---|---|---|---|---|---|
| | | | | | Major solute | internal aqueous |
| | | | Volume-to- | | and its | phase solution to |
| | | | molar mass | | concentration | organic phase |
| | PEI | | ratio of | | of external | solution to |
| Item | concentration | PLGA | PEI to | Organic | aqueous phase | external aqueous |
| number | (v/v) | concentration | siRNA | phase | solution | phase solution |
| A1 | 10% | 5 mg/mL | 200 mL:2.5 | Chloroform | PEG 10 | 1:5:15 |
| | | | μmol | | mg/mL | |
| A2 | 10% | 5 mg/mL | 200 mL:2.5 | Chloroform | Tween 80 10 | 1:5:15 |
| | | | μmol | | mg/mL | |
| A3 | 10% | 5 mg/mL | 200 mL:2.5 | DMF | PEG 10 | 1:5:15 |
| | | | μmol | | mg/mL | |
| A4 | 10% | 5 mg/mL | 200 mL:2.5 | DMF | Tween 80 10 | 1:5:15 |
| | | | μmol | | mg/mL | |

### 2. Encapsulation Efficiency of Nanocomplex

The synthesized nanocomplex was centrifuged under specific conditions, followed by washing with distilled water and PBS respectively. The washed products (suspensions) were detected by using a nanoflow cytometer to compare the siRNA encapsulation efficiencies of the two washed products.

3. The effect of the nanocomplex on the hatching of fertilized eggs: the nanocomplex was suspended in PBS, and Epinephelus lanceolatus fertilized eggs were subjected to medicated bath. The groups were divided as follows: Ctr: control group, C1: 10-fold dilution concentration, and C2: 1000-fold dilution concentration. The hatching rate was observed and calculated.

### 4. Results and Analysis

### 4.1 the Effect of Different Groups on Particle Size

### Group A1 Chloroform-PEG:

An organic phase chloroform and an external aqueous phase PEG were functionally synthesized and then detected. The particle size of the product subjected to secondary ultrasonic treatment was 156.5 nm. The light intensity distribution was at 200 nm.

### Group A2 Chloroform-Tween 80:

An organic phase chloroform and an external aqueous phase Tween 80 were functionally synthesized and then detected. The particle size of the product subjected to secondary ultrasonic treatment was 146 nm. The light intensity distribution was at 200 nm.

### Group A3 DMF-PEG:

An organic phase DMF and an external aqueous phase PEG were functionally synthesized and then detected. The particle size of the product subjected to secondary ultrasonic treatment was 189.2 nm. The light intensity distribution was at 200 nm.

### Group A4 DMF-Tween 80:

An organic phase DMF and an external aqueous phase Tween 80 were functionally synthesized and then detected. The particle size of the product subjected to secondary ultrasonic treatment was 79.53 nm, and light intensity distribution showed double peaks.

The comparison results are as follows: in a relationship graph of a particle size and light intensity, A1, A2 and A3 are all single peaks and their peak values are concentrated at about 200 nm; and A4 is double peak, indicating that the formed nanoparticle has an non-uniform structure.

4.2 Stability of Nanoparticles after Logarithmic Serial Dilution of Four Formulations

The nanoparticles prepared in Groups A1-A4 were subjected to 2⁰ to 2⁹-fold dilution, and the maximum ratio at which the final product remained stable was investigated. The results are shown in Table 3 below.

TABLE 3

| Dilution | | Particle | | Particle | | Particle | | Particle |
|---|---|---|---|---|---|---|---|---|
| fold | A1 | size/nm | A3 | size/nm | A2 | size/nm | A4 | size/nm |
| 2⁰ | Single | 184.4 | Single | 243.8 | Double | 301 | Double | 525.7 |
| | peak | | peak | | peak | | peak | |
| 2¹ | Double | 152.5 | Double | 316.1 | Triple | 280.3 | Double | 383.4 |
| | peak | | peak | | peak | | peak | |
| 2² | Double | 227.1 | Double | 383.2 | Double | 529.6 | Multiple | 530.6 |
| | peak | | peak | | peak | | peak | |
| 2³ | Single | 176.9 | Single | 414.4 | Triple | 807.2 | Single | 806.9 |
| | peak | | peak | | peak | | peak | |
| 2⁴ | Double | 326.5 | Double | 455.7 | Single | 657.7 | Single | 604 |
| | peak | | peak | | peak | | peak | |
| 2⁵ | Single | 358 | Single | 621.5 | Double | 934.1 | Single | 745.5 |
| | peak | | peak | | peak | | peak | |
| 2⁶ | Single | 522.3 | Double | 556.7 | Single | 905.9 | Single | 757.5 |
| | peak | | peak | | peak | | peak | |
| 2⁷ | Single | 609 | Single | 867.6 | Double | 679.9 | Single | 1251 |
| | peak | | peak | | peak | | peak | |
| 2⁸ | Single | 858.4 | Double | 1184 | Double | 835.6 | Multiple | 30.02 |
| | peak | | peak | | peak | | peak | |
| 2⁹ | Single | 631.6 | Single | 785 | Single | 1613 | Single | 1416 |
| | peak | | peak | | peak | | peak | |

By comparing chloroform with DMF, the results showed that the nanostructure formed when chloroform was used as an oil phase was uniform (single peak), and the particle size exceeded 300 nm upon 2⁻⁴-fold dilution, whereas the particle size exceeded 300 nm upon 2⁻¹-fold dilution when DMF was used as the oil phase; and chloroform was selected as the component of the oil phase since the objective of this experiment was to form smaller particles to facilitate the entry of nanoparticles into fertilized eggs. When PS80 was used as the external aqueous phase, the nanoparticle size upon 21 dilution exceeded 300 nm, and the structure of the nanoparticle formed when Tween 80 was used was non-uniform (double peak); and when PEG was used as the external aqueous phase, the formed nanoparticle had the particle size of less than 300 nm and a uniform structure, and therefore PEG was selected as the external aqueous phase. Thus, chloroform-PEG was selected as optimal reagents for the optimal organic phase and the external aqueous phase.

### 4.3 Material Parameters in Optimal Ratio

After determining the optimal reagent combined with the optimal ratio (the volume ratio of the internal aqueous phase solution to the organic phase solution to the external aqueous phase solution was 1:5:15), the particle size and zeta potential of the product subjected to secondary ultrasonic treatment were measured following synthesis and modification according to operation steps for synthesizing the PLGA-siRNA nanocomplex. The sufficiently washed and dried product was observed using an electron microscope.

### (1) the Particle Size and Zeta Potential Results of a Solution Subjected to Secondary Ultrasonic Treatment

The solution subjected to secondary ultrasonic treatment obtained using an optimal reagent in an optimal ratio had a nanoparticle size of 109.7 nm and a zeta potential of 30.91 mV, with light intensity distribution being a single peak.

### (2) the Particle Size and Zeta Potential Results of an Ultrafiltration Centrifugation Solution

The solution subjected to secondary ultrasonic treatment obtained using an optimal reagent in an optimal ratio had a nanoparticle size of 15.78 nm and a zeta potential of 5.075 mV, with light intensity distribution being a single peak.

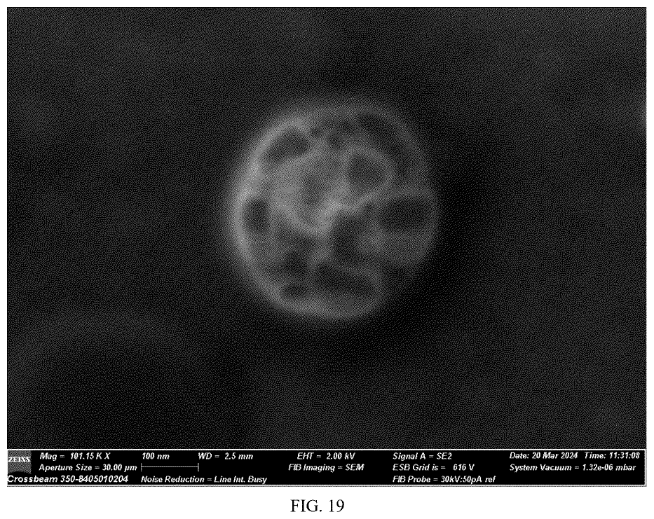

(3) the Particle Size and Zeta Potential of an Ultrafiltration Membrane Washing Solution and Electron Microscope Observation of a Final Dried Product The ultrafiltration membrane washing solution of the solution subjected to secondary ultrasonic treatment obtained using an optimal reagent in an optimal ratio had a nanoparticle size of 106.7 nm and a zeta potential of 32.82 mV, with light intensity distribution being a single peak. The filter membrane washing solution was observed under the electron microscope after drying, as shown in FIG. 19, indicating that obvious spherical particles occurred under the proportion scale of 100 nm. By comparing the distilled water washing effect with the PBS washing effect, the results showed that after PBS washing, the nanoparticle was more stable, and its particle size was more concentrated and was centered around 160 nm. The results are basically consistent with those of the nanoparticle laser particle size analyzer and electron microscope structures.

There may be certain deviations in particle sizes measured using different instruments, which belongs to a normal phenomenon.

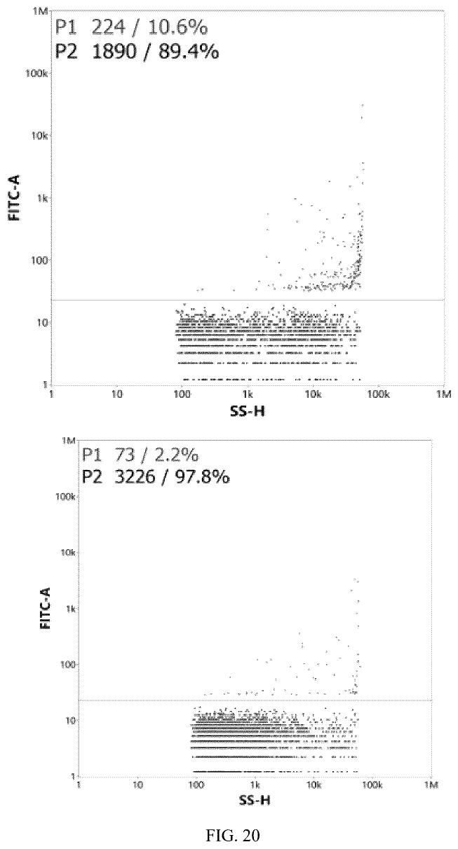

(4) The encapsulation efficiencies of the nanomaterials on siRNA: the nanocomplex was subjected to flow cytometric fluorescence detection after being suspended using purified water and PBS. The results are as shown in FIG. 20, it is found that the encapsulation efficiency of siRNA in PBS is more stable and higher than that in water.

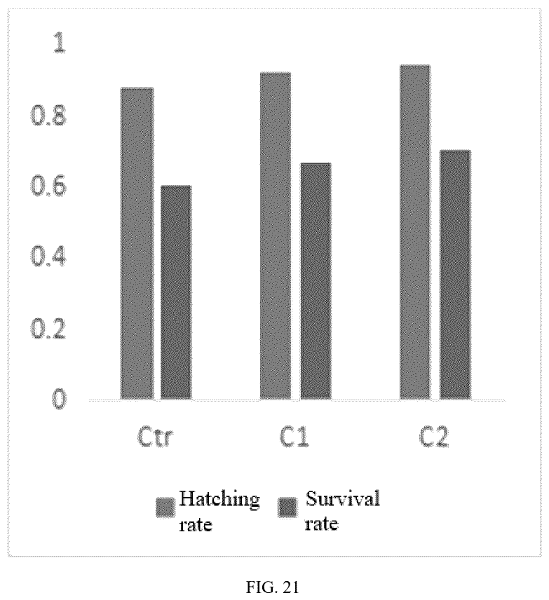

(5) The effect of the nanocomplex on the hatching of Epinephelus lanceolatus fertilized eggs: the nanocomplex subjected to PBS suspension was used as a parent source, the diluted suspension was respectively diluted by 10-fold and 1000-fold, then the Epinephelus lanceolatus fertilized eggs were subjected to medicated bath for 1-2 h. The results are as shown in FIG. 21, showing that the hatching rates were respectively 92% and 94%, and the hatching rate of the control group was 88%, which indicates that this material has no toxic effects on the Epinephelus lanceolatus fertilized eggs. Within a period of time after hatching, the survival rates were respectively 66% and 70%, and the survival rate of the control group was 60%, indicating that the survival rates of the medicated bath group are higher than those of the control groups. Based on the above results, the nanocomplex exhibits no toxic effects on the Epinephelus lanceolatus fertilized eggs and can increase their survival rates.

The above descriptions are only preferred embodiments of the present disclosure. It should be noted that for those of ordinary skill in the art, several improvements and modifications may be made without departing from the principle of the present disclosure, and these improvements and modifications should be also deemed to be included within the protective scope of the present disclosure.

What is claimed is:

1. A nanocomplex, wherein the nanocomplex is composed of the following raw materials: poly(lactic-co-glycolic acid), polyethyleneimine and siRNA, wherein
a mass-to-volume ratio of the poly(lactic-co-glycolic acid) to the polyethyleneimine is (5-50) mg:(0.05-1) mL, and each 100 mL of the polyethyleneimine contains 0.5-2.5 μmol of siRNA; and
the siRNA comprises at least one of a positive-sense strand of a nucleotide sequence as shown in SEQ ID NO: 1 and a negative-sense strand of a nucleotide sequence as shown in SEQ ID NO: 2, a positive-sense strand of a nucleotide sequence as shown in SEQ ID NO: 3 and a negative-sense strand of a nucleotide sequence as shown in SEQ ID NO: 4, and a positive-sense strand of a nucleotide sequence as shown in SEQ ID NO: 5 and a negative-sense strand of a nucleotide sequence as shown in SEQ ID NO: 6; and the nanocomplex has a particle size of 100-200 nm.

2. The nanocomplex according to claim 1, wherein the mass-to-volume ratio of the poly(lactic-co-glycolic acid) to the polyethyleneimine is 25 mg: 0.1 mL, each 100 mL of the polyethyleneimine contains 1.25 mol of siRNA, wherein the molecular weight of the poly(lactic-co-glycolic acid) is 10000 Da, and the molecular weight of the polyethyleneimine is 25000 Da.

3. The nanocomplex according to claim 1, wherein the siRNA comprises the positive-sense strand of the nucleotide sequence as shown in SEQ ID NO: 1 and the negative-sense strand of the nucleotide sequence as shown in SEQ ID NO: 2; and the nanocomplex has the particle size of 160 nm.

4. A preparation method of the nanocomplex according to claim 1, comprising the following steps:
Step 1, mixing the siRNA with the polyethyleneimine to obtain a solution A, and mixing poly(lactic-co-glycolic acid) with an organic phase solution to obtain a solution B;
Step 2, performing ultrasonic mixing on the solution A and the solution B in Step 1;
Step 3, performing ultrasonic mixing on the solution subjected to ultrasonic mixing in Step 2 and an external aqueous phase solution again; and
Step 4, performing filtration and centrifugation on the solution subjected to ultrasonic mixing again in Step 3 so as to obtain the nanocomplex.

5. The preparation method according to claim 4, wherein in the Step 1, a solvent used for mixing the solution A comprises water, and the organic phase solution comprises chloroform and/or dimethylformamide; and
the concentration of the polyethyleneimine in the solution A is 5 vol %-50 vol %, and the concentration of the poly(lactic-co-glycolic acid) in the solution B is 1-10 mg/mL.

6. The preparation method according to claim 4, wherein the organic phase solution is chloroform, the concentration of the polyethyleneimine in the solution A is 10 vol %, and the concentration of the poly(lactic-co-glycolic acid) in the solution B is 5 mg/mL.

7. The preparation method according to claim 4, wherein in the Step 3, the solute of the external aqueous phase solution comprises polyethylene glycol and/or Tween 80.

8. The preparation method according to claim 4, wherein the solute of the external aqueous phase solution is polyethylene glycol.

9. The preparation method according to claim 4, wherein the concentration of polyethylene glycol in the external aqueous phase solution is 1-20 mg/mL.

10. The preparation method according to claim 4, wherein the concentration of polyethylene glycol in the external aqueous phase solution is 10 mg/mL.

11. The preparation method according to claim 4, wherein in the Step 2 and Step 3, a volume ratio of the solution A to the solution B to the external aqueous phase solution is (0.5-2):(1-10):(1-30).

12. The preparation method according to claim 4, wherein in the Step 2 and Step 3, the volume ratio of the solution A to the solution B to the external aqueous phase solution is 1:5:15.

13. The preparation method according to claim 4, wherein in the Step 4, the filtration adopts ultrafiltration, and after the centrifugation, the Step 4 further comprises washing with phosphate buffer saline (PBS) to obtain the nanocomplex.

14. A drug for resisting a nervous necrosis virus (NNV) and/or improving the survival rate of fish eggs infected with NNV, comprising the nanocomplex according to claim 1.

15. A method for improving the survival rate of fish eggs infected with NNV, comprising performing medicated bath on the fish eggs using the drug according to claim 14.
